(19) United States
(12) Patent Application Publication
(10) Pub. No.: US 2004/0126755 A1
(43) Pub. Date: Jul. 1, 2004

(54) Gene expression based method for distinguishing metastatic from non-metastatic forms of a tumor, and use in designing therapeutic drugs

(76) Inventors: Dietrich A. Stephan, Falls Church, VA (US); Tobey J. MacDonald, Washington, DC (US); Kevin M. Brown, Washington, DC (US)

Correspondence Address: Dr. Melvin Blecher, Second Floor, 4329 Van Ness St., NW, Washington, DC, 20016-5625, US

(21) Appl. No.: 09/940,454

(22) Filed: Aug. 29, 2001

(51) Int. Cl. C12Q 1/68

(52) U.S. Cl. 435/6

(57) ABSTRACT

Gene expression profiling of tumors, clinically designated as either metastatic (M+) or non-metastatic (M0), identifies genes whose expression differed significantly between classes. A class-prediction algorithm based on these medulloblastoma genes assigned the sample class to these tumors (M+ or M0) with 72% accuracy and to four additional independent tumors with a 100% accuracy. Class prediction also assigned the metastatic medulloblastoma cell line Daoy to the metastatic class. Notably upregulated in the M+ tumors were platelet derived growth factor receptor alpha (PDGFRA) and members of the downstream RAS/mitogen-activated protein kinase (MAPK) signal transduction pathway. Immunohistochemical validation on an independent set of tumors showed significant overexpression of PDGFRA in M+ tumors as compared to M0 tumors. In in vitro assays, PDGFA enhanced medulloblastoma migration and increased downstream MAP2K1 (MEK1), MAP2K2 (MEK2), MAPK1 (p42 MAPK), and MAPK3 (p44 MAPK) phosphorylation in a dose-dependent manner. Neutralizing antibodies to PDGFRA or U0126, a highly specific chemical inhibitor of MAP2K1 and MAP2K2 known as U0126, blocked MAP2K1, MAP2K2, and MAPK1/3 phosphorylation, inhibited migration, and prevented PDGFA-stimulated migration. These results provide the first insight into the genetic regulation of medulloblastoma metastasis and are the first to suggest a role for and the RAS/MAPK signaling pathway in medulloblastoma metastasis. Inhibitors of PDGFRA and RAS proteins, among others overexpressed M+ genes identified herein, represent novel therapeutic targets in medulloblastomas and other M+/MO tumors. The inventive method of prediction and targeted therapy is applicable to any tumor that exists in both M+ and MO forms, such as the neurotumors glioma, neuroblastoma and ependymoma, as well as lung and breast cancers.

### BACKGROUND OF THE INVENTION

The invention is directed to prognostic method for distinguishing metastatic from non-metastatic forms (classes) of a tumor, and also to the use of such methods to identify target genes for therapeutic intervention. More specifically, the method is directed to the development class prediction algorithms based on genes identified by expression profiling of tumor tissue, and to the use of such algorithms to distinguish metastatic from non-metastatic forms of a tumor. The metastatic genes thus identified are used to design drugs that are therapeutic against metastasizing tumors.

Highly malignant primitive neuroectodermal cerebellar tumors of children, such as medulloblastoma, are brain tumors in which treatment intensification is based in part on the presence or absence of metastasis at diagnosis. The tendency of such tumors to disseminate throughout the central nervous system early in the course of illness complicates its treatment. Despite the use of whole brain and spine radiation for the prevention and treatment of dissemination, nearly half of the patients will die of early tumor recurrence and leptomeningeal spread while the majority of survivors will suffer significant neurocognitive sequelae as a result of this therapy. The latest Children?s Cancer Group report of 188 patients with medulloblastoma identified disseminated disease as the most powerful independent factor associated with poor survival (P=0.0006). Clearly, a better understanding of the regulation of metastasis is necessary for the discovery of novel therapeutic targets and to facilitate a better assessment of individual risk for tumor progression.

Although histologic features have failed to provide an accurate prediction of clinical-biological behavior in medulloblastoma, the genetic events that play a role in the biology of these tumors may allow for molecular subtyping. Genetic insight could lead to the development of novel therapeutic strategies selectively directed against promoters of dissemination. Yet, only a few genetic alterations have been associated with biological behavior in medulloblastoma. Grotzer et al. J. Clin Oncol. 18:1027 (2000) reported that high expression of the neurotrophin receptor NTRK3 mRNA (also known as TrkC) is an independent predictor of favorable outcome (P<0.00005). Subsequent studies showed that NTRK3 mediates apoptosis in the presence of NTRK3 ligand, neurotrophin-3 (NTF3), by inducing immediate-early gene expression of JUN and FOS (Packer, Brain Develop. 21:75 (1999). Herms et al. described MYC mRNA expression in 42% of medulloblastomas tested, which correlated with poor survival (P=0.02) (Int. J. Cancer 89:395 (2000)). Less clear is the significance of chromosome 17p in medulloblastoma. Although 17p deletions occurred 25-50% of cases, a putative tumor suppressor gene remains unidentified. Batra et al.J Neurooncol. 24:39 (1995) reported that 17p deletions were associated with a shortened survival period (P=0.045); however, studies by Biegel et al. Clin. Cancer Res. 3:473 (1997) and Emadian et al Clin Cancer Res. 2:1559 (1996) showed no such association, and maintained that clinical risk factors such as metastasis at diagnosis remained better prognostic indicators. Whether NTRK3 or MYC is involved in metastasis regulation is unknown, and until the present invention, no specific pattern of genetic alterations has been linked to metastasis in cancers such as medulloblastoma.

For metastasis to occur after the onset of oncogenesis, cells must migrate away from the primary tumor, invade through connective tissue, implant in a distant site, and re-establish a blood supply. Identification of the factors regulating the coordination of such responses has not been amenable to study through a gene by gene approach due to the complexity of the system. In addition, the traditional non-hypothesis driven techniques of human genetics, such as positional cloning, are not well suited to identifying these somatic events. Recently, expression array profiling, whereby one studies the steady-state levels of thousands of genes in cells of interest in parallel, has become the ideal tool for this type of analysis.

Golub et al. Science 286:531 (1999) applied the technique to distinguish between cancer subtypes. The authors distinguished acute lymphoblastic leukemia (ALL) from acute myelogenous leukemia (AML) based strictly on gene expression profiles. Prediction of mestastases was not addressed. Bittner et al. Nature 406:536 (2000) tested the resolution of the array-based strategy to dissect out more subtle differences in a study of melanomas in various stages of progression. In this study, a cDNA array-based strategy was able to detect a set of genes that behaved differently in metastatic melanoma in comparison to lesions that had not yet started to disseminate, despite each group displaying identical histologic characteristics. Bittner et al. showed that the metastatic tumors had acquired a somatic potential as they progressed. These findings were validated using blocking antibodies to the protein target of a selected gene unique to the metastatic class resulting in the inhibition of in vitro scratch healing.

Until the present invention, a genetic method for distinguishing metastatic from non-metastatic tumors, e.g., medullablastomas, was unknown. Clearly, such a method would be highly desirable clinically, not only in managing the patient, but also as a prognostic model for drugs designed to treat the disease. Such a method has been now discovered, and is described in detail below.

### SUMMARY OF THE INVENTION

The invention in concept is a gene expression-based method for predicting to a high degree of certitude metastasis in human tumors that exist in both metastatic (M+) and non-metastatic (MO) classes (forms). In reduction to practice, the invention is comprised of three general steps. In the first step, the technique of expression-profiling of the M+ and M 0 subclasses of a series of reference tumors of interest, together with permutational statistical analyses, is used to identify dysregulated genes whose expression differ statistically and significantly between the two forms of a tumor, and that are upregulated (i.e., overexpression) in the M+ class. From this information, a class-predictive algorithm based upon said predictive genes is derived. In the last step, this algorithm is then applied to produce Prediction Strength values ranging between 0 and 1 that are used to assign sample classes (M+ or M0) to a candidate tumor.

In one embodiment of the invention, the upregulated M+ genes thus identified are used as therapeutic targets, that is to say, specific drugs are designed either to downregulate the gene of interest or to prevent its upregulation. M+ genes that are of particular value in this therapeutic regard are the PDGFRA and RAS/MAPK families of genes, although any of the 85 M+ genes identified in this invention would also be suitable therapeutic targets.

### DESCRIPTION OF THE DRAWINGS

FIG. 1 Genes differentiating metastatic from non-metastatic tumors in 23 individuals arrays. Average intensities of both tumor classes, permutational P values, and average fold-differences are presented for all statistically significant probe sets. Varying levels of expression are represented on a scale from dark green (lowest expression) to bright red (highest expression).

FIG. 2 Classification and prediction strengths of non-metastatic tumors (♦), metastatic tumors (▾), non-metastatic validation tumors (▪), and medulloblastoma cell lines (●). Prediction strengths range between 1 (high confidence) and 0 (low confidence). Samples with a prediction strength value below 0.23 are considered to be uncertain and are not classified.

FIG. 3 Protein expression validates microarray results. Immunohistochemistry was performed for PDGFRA, SPARC, and PCNA in an independent set of metastatic (M+) and non-metastatic (M0) medulloblastomas, photographed by light microscopy at 200X. Each of the differential gene products identified by immunoperoxidase staining is overexpressed by M+ vs. M0 tumors. Three representative tumors for each tumor class are presented. See Table 1 for a summary of immunohistochemistry results.

FIG. 4 PDGFRA promotes medulloblastoma cell adhesion. Highly metastatic Daoy cells were allowed to adhere to fibronectin-coated wells in the presence of increasing concentrations of PDGFA and after pre-treatment with neutralizing monoclonal antibody to PDGFRA. Each bar represents the mean +/− s.e.m of duplicate experiments.

FIG. 5 PDGFRA promotes medulloblastoma cell chemotaxis. a, Neutralizing antibody to PDGFRA inhibits PDGFA-mediated cell migration. Daoy cells were pre-incubated with either a neutralizing antibody to PDGFRA, an isotype-matched control antibody, or no antibody, and subsequently induced to migrate towards increasing concentrations of PDGFA. Each bar represents the mean “s.e.m of duplicate experiments. b, Inhibition of MAP2K1 and MAP2K2 blocks PDGFA-mediated cell migration. Daoy cells were induced to migrate towards increasing concentrations of PDGFA following pre-treatment with MAP2K1/2 inhibitor. Each bar represents the mean +/− s.e.m of duplicate experiments.

FIG. 6 PDGFRA blocking antibody inhibits activation of MAPK pathway members. a, PDGFRA blocking antibody inhibits PDGFA-mediated MAP2K1/2 phosphorylation. Daoy cells were incubated with PDGFA following incubation with either PDGFRA antibody or the MAP2K1/2 inhibitor U0126. Lysates were subjected to SDS-PAGE followed by Western blotting with antibodies against MAP2K1/2 and phospho-MAP2K1/2. The ratio of phspho-MAP2K1/2 to total MAP2K1/2 was used to determine relative change in phosphorylation. b, PDGFRA blocking antibody and MAP2K1/2 inhibitor blocks PDGFA-mediated MAPK1/3 phosphorylation. Cells were treated as above. Western blots used antibodies against MAPK1/3, and phospho-MAPK1/3. The ratio of phospho-MAPK1/3 to total MAPK1/3 for each lane was used to determine the relative change in phosphorylation.

FIG. 7 Schema of PDGFRA_activation of the RAS/MAPK pathway. Diagram indicates the identification of all differentially expressed genes (red text label) in metastatic (M+) vs. non-metastatic (M0) medulloblastomas that have been previously characterized as having functions within the RAS/MAPK signal transduction pathway. Upregulated (↑) and downregulated (↓) genes by M+ vs. M0 tumors are noted. Black arrows represent reactions; blue arrows represent catalysis of a reaction, and green arrows represent activation of one protein by another.

### DESCRIPTION OF THE PREFERRED EMBODIMENTS

We describe below the use of oligonucleotide expression-profiling to identify genes prognostic of metastasis of certain tumors, and we describe how such genes may serve as new therapeutic targets for the creation of “smart drugs”.

A cohort of frozen tumors can be used for gene expression profiling, along with permutational statistic analyses, as described in detail in the examples below, to identify sets of genes that are significantly differentially expressed between metastatic (M+) and non-metastatic (M0) samples. These predictor gene sets so identified can be used accurately in a derived algorithm to classify additional tumors with blinded M-status, as well as correctly assign M+ status to a metastatic cell line. The results can be further validated by immunostaining an independent tumor set.

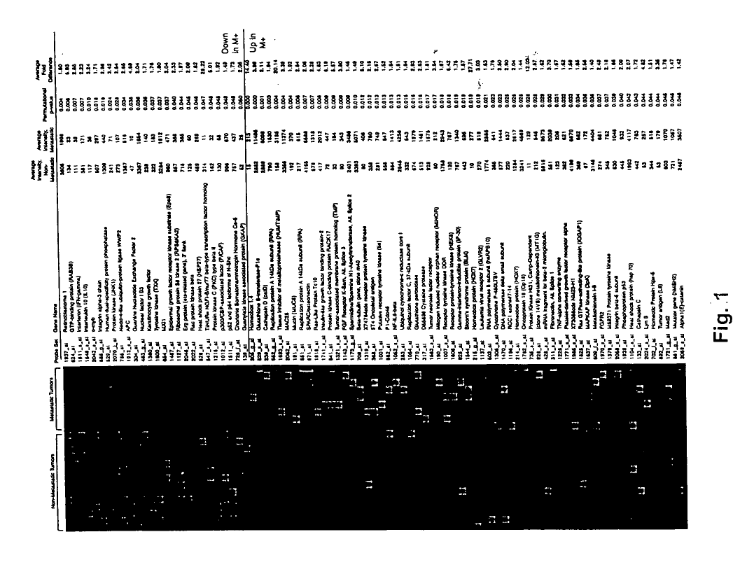

FIG. 1 provides a gene list containing 85 genes, 59 of which are enhanced, i.e., upregulated, in M+ medulloblastomas. The platelet derived growth factor receptor alpha (PDGFRA) and multiple members of the downstream RAS/MAPK signal transduction pathway were notably upregulated in metastatic medulloblastomas.

Blocking upregulation of a gene upregulated in M+ subclass tumors can be used to determine if the effects of upregulation can be reversed. For example, we found that inhibiting PDGFRA upregulation decreases MAP2K1, MAP2K2, MAPK1, and MAPK3 activation and inhibits the metastatic potential of Daoy medulloblastoma cells in vitro, thereby revealing a novel therapeutic target in this tumor type. A variety of inhibitors, including chemicals and blocking antibodies, are effective in this regard. Inhibitors and their sources are described at the end of this section, in the examples below, and in the claims.

Using oligonucleotide expression profiling, as noted above, a set of 85 medulloblastoma candidate predictor genes were identified whose expression differed significantly between metastatic and non-metastatic forms of the tumor. This set of genes allowed us to assign sample class to the original tumor set with 72% accuracy using a leave-one-out approach, as well as to four additional independent tumors with 100% accuracy. Expression results at the protein level can be verified via standard immunohistochemistry techniques in an independent tumor set, and subsequently by in vitro adhesion and migration functional assays, all by standard techniques outlined below. As mentioned above, a number of dysregulated genes are notably involved in the PDGFRA and downstream RAS/MAPK signaling pathway and may thus serve as exploitable targets for directed therapeutics.

Using our set of class-correlated predictor genes, 72% of our classifications (13/18) were correct using a leave-one-out approach. Using a gene list generated from all 23 samples, we accurately assigned four of five additional blinded medulloblastomas, not used in building our initial predictor, to their appropriate class with no mis-predictions. The single non-metastatic tumor mispredicted with a high degree of confidence was the only sample without clear radiographic validation of staging.

As noted above, protein validation of the array results can be demonstrated by standard immunohistochemistry tests. For example, we found (see below) that two key proteins that were found over-expressed in M+ tumors, PDGFRA and SPARC, showed up in a significantly higher percentage of M+ tumors staining positive compared to M0 tumors (P=0.0004 and 0.0025, respectively).

We identified 85 dysregulated predictor genes, 59 of which (M+) were shown to play a role in at least one of the following functional categories:

1. Invasion and angiogenesis (15 genes)

2. Growth factor or cytokine-mediated proliferation (12 genes)

3. Signal transduction (9 genes)

4. Transcriptional regulation (8 genes)

5. DNA replication (8 genes)

6. Oncogenesis (7 genes).

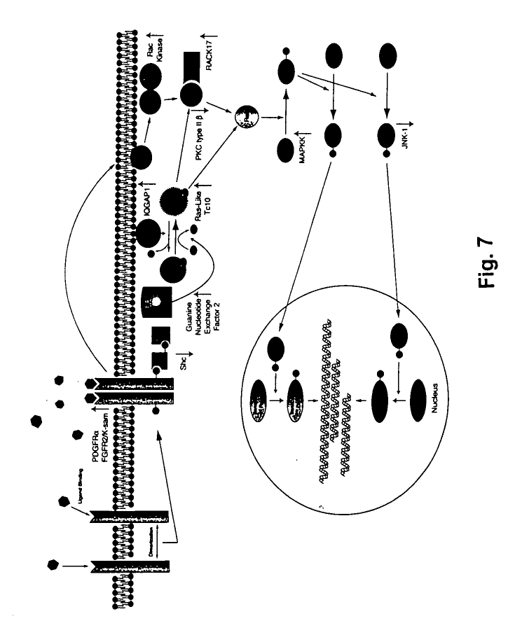

Furthermore, the majority of members within each functional grouping interact directly with one another or share a common pathway, such as the DNA replication genes, DNA polymerase delta subunit, replication factor C, and replication protein A. Importantly, a significant number of differentially expressed genes (71%) uncovered by this study are newly described in medulloblastoma. These notably include the genes HOXA4 and HOXA7, cathepsin C (CTSC) and D (CTSD), Bloom syndrome protein (BLM), trophoblast glycoprotein (TPBG), MSH2, and platelet derived growth factor receptor alpha (PDGFRA). Particularly striking is the over-expression of the RAS/MAPK signaling pathway in M+ tumors, in which all nine genes identified as mediators of signal transduction are members of this pathway (FIG. 7).

Prior to the present discovery, no consistent genetic alteration or pattern has been linked to medulloblastoma metastasis. Clark et al. Nature 406:532 (2000), using Affymetrix oligonucleotide array expression profiling in melanoma, demonstrated that RhoC (ARMC), encoding a cytoskeletal organizing protein, plays an essential role in the complex series of events leading to metastasis. Consistent with this finding is the presently observed over-expression of α1-E catenin, a separate cytoskeletal organizing protein also found upregulated in the study above, and decreased expression of RAB3B, an actin-regulating GTPase protein, by M+ tumors. Together, these findings suggest that cytoskeletal regulation is critical for cell motility. Other metastasis-promoting genes identified in the melanoma study of Clark et al., that are also upregulated in our series of M+ tumors, include RAS GTPase activating-like gene (IQGAP1), heat shock protein 70, and fibronectin. Fibronectin is particularly interesting since its expression has been linked with the promotion of metastasis, possibly by serving as a common ligand for the integrin family of cell-matrix adhesion receptors. Indeed, Yi et al. PNAS 98:620 (2001) demonstrated that treatment of tumors with polymeric fibronectin inhibited angiogenesis and metastasis in vivo.

A number of differentially expressed genes in the present study have been associated with the promotion of metastasis through cell-matrix interactions related to invasion and angiogenesis. These include M+ tumor down regulation of integrin α3 (ITGA3) and upregulation of integrin α5 (ITGB5), SPARC, TIMP1, and the angiopoietin receptor TIE. Integrins control the balance between adhesion and detachment required for cell migration. Thus, concomitant downregulation of ITGA3 and upregulation of ITGB5 may allow M+ tumors to disseminate more readily. SPARC, an anti-adhesive and pro-angiogenic matricellular glycoprotein, was also shown by Thomas et al. Clin. Cancer Res. 6:1140 (2000) to directly correlate with progression and metastasis of prostate cancer. TIMP1, an inhibitor of matrix metalloproteinases, is generally considered inhibitory of metastasis; however, Remacle et al. Int. J. Cancer 89:118 (2000) reported that high levels of TIMP1 expression correlate with breast cancer progression, suggesting that it may have other tumor-promoting function. The angiopoietin tyrosine kinase receptor gene TIE has been described in the regulation of vasculogenesis during ischemia and hypoxia, conditions commonly observed during rapid medulloblastoma growth.

Interestingly, M+ tumors also overexpress two members of the HOX family of genes (HOXA4 and HOXA7). Cillo et al., Int J. Cancer 66:692 (1996) demonstrated that HOX gene expression correlated inversely with the ability of clonal melanoma populations to form cell-cell and cell-matrix interactions (i.e. can achieve metastatic potential). The fact that both serine and matrix metalloproteinases, typically considered to promote metastasis, are absent from our gene list suggests that activation of these targets may be controlled by upstream genetic events. Alternatively, genes regulating metastasis may be specific to histologic type rather than a universal phenotype. Genes that have been previously associated with outcome in medulloblastoma were not differentially expressed between M+ and M0 tumors, but showed the following profiles: NTRK3 (present in 39% of tumors tested) and MYC (61%). These findings suggest that the relationship of NTRK3 and MYC expression may be more due to biological response to radiation and chemotherapy. Genes previously reported to have altered expression in medulloblastoma, such as MYCN (present in 35% of tumors tested), PTCH tumor suppressor gene (22%), and ERBB2 (0%), were not differentially expressed.

Cell growth is one of the basic steps leading to metastasis. Growth factors and their receptors are prominent on our list of upregulated genes by M+ tumors in Table 1. These include the receptors for platelet derived growth factor alpha (PDGFRA) and fibroblast growth factor (FGFR2), as well as two members of the insulin-like growth factor family (IGFBP2 and IGFBP7). Gene expression profiling of low-grade gliomas by Huang et al. (above) also demonstrated over-expression of PDGFRA, as well as SPARC and TIMP1 by infiltrative tumor. PDGFRA, a promoter of angiogenesis, was the only overexpressed M+ gene in our study that was also found increased in a study by Markert et al. (2001, above) comparing highly invasive glioblastoma multiforme tumors to normal brain. Cells in the early oligodendrocyte lineage express PDGFRA during proliferation and CNS organization, and PDGFA is a key regulatory factor for glial differentiatio. Thus, it is possible that PDGFRA expression in M+ medulloblastomas may represent the acquisition of more glial characteristics. PDFGRA induces cell migration through interaction with focal adhesion kinase (PTK2) and integrins and can independently activate the RAS signal cascade regulating mitogenesis. Dysregulation of multiple members of the RAS/MAPK pathway by M+ tumors in this study suggests that PDGFRA/RAS/MAPK may be a dominant pathway leading to tumor metastasis. M+ tumors also show downregulation of MAPK8, a transcription factor downstream in the MAPK pathway, which has been shown to induce apoptosis and PC12 cell differentiation in response to nerve growth factor.

Having uncovered a potential role for PDGFRA and downstream activation of the RAS/MAPK signaling pathway in medulloblastoma metastasis (FIG. 7), we tested whether the invasive potential of Daoy metastatic medulloblastoma cells is inhibited by blocking PDGFRA. We chose the Daoy medulloblastoma cell line because it appears to closely mimic the in vivo expression profiles of human medulloblastomas. Daoy cells express 97.6% of the genes consistently expressed above the detection threshold in medulloblastoma tumors (genes called “present” by the Affymetrix software in 100% of the medulloblastomas profiled). These cells also fail to express 96.6% of the genes consistently not expressed in medulloblastoma (expression below the threshold of detection in 100% of the tumors). Additionally, of the cell lines profiled, expression levels of the 85 “predictor” genes in Daoy most closely resembled that of the metastatic tumors. We showed that PDGFA stimulates fibronectin-directed migration of Daoy in a dose-dependent manner, and that blocking PDGFRA with a neutralizing antibody reduces adhesion and specifically blocks the PDGFA dose-dependent response in migration across fibronectin-coated membranes. The mechanism behind the exaggerated antibody-induced reduction in cell migration we observed at higher concentrations of PDGFA is unclear. We speculate that saturation of PDGFRA with PDGFA ligand and the PDGFRA blocking antibody may induce internalization and degradation of the receptor in endosomes, resulting in a greater blockade of PDGFRA mediated signal. Further studies will be necessary to confirm this hypothesis. The reduction in cell adhesion to fibronectin-coated plates by blocking antibodies to PDGFRA suggests that cell-surface PDGFRA may act to bind fibronectin directly, whereas PDGFRA activation by PDGFA ligand enhances motility. A critical balance between these two functions may be essential for the coordination of migration across fibronectin.

We additionally demonstrated that PDGFA stimulation of medulloblastoma cells enhances the phosphorylation of downstream MAP2K1, MAP2K2, and MAPK1/3, while blocking antibodies to PDGFRA inhibits phosphorylation. Thus, PDGFA-stimulated migration of medulloblastoma cells directly correlates with downstream MAP2K1, MAP2K2, and MAPK1/3 activation. We showed that the highly specific MAP2K1/MAP2K2 inhibitor U0126 (extracellular signal-regulated protein kinase inhibitor, obtainable from Cell Signaling Technology, Inc., 166B Cummings Center, Bldg. 100, Beverly, Mass. 01915; also reduces phosphorylation of MAPK1/3 and similarly inhibits the migration of medulloblastoma cells across fibronectin). Specific inhibitors of PDGFRA, such as the novel signal transduction inhibitor STI-571 (obtainable as GLEEVEC (™) from Novartis Pharmaceuticals Corp., US Rte. 59, East Hanover, N.J. 07936) or against RAS, such as the currently available agents R115777 (a farnesyl transferase inhibitor, available from Janssen Pharmaceutical Inc., Titusville, N.J. 08560), SCH66336 (obtainable from Schering-Plough Research Institute, Kenilworth, N.J. 07033-1300), may also be used as treatments against targeted overexpressed genes in metastasizing tumors, including medulloblastomas. In addition, other agents targeting overexpressed metastatic genes include: wortmannin (a phosphoinositide 3-kinase inhibitor, available from Eli Lilly, Indianapolis, Ind.), NM3 (that targets the FGF receptor and VEGF expression, available from ILEX, Inc.), CC1-779 (works against MAP kinase, available from Wyeth/American Home Products), and TLK 286 (targets glutathione-S-transferase gene, available from Telik, So. San Francisco, Calif.).

While the medulloblastoma embodiment figures prominantly in this application, it should not be assumed that the present invention is limited to this kind of tumor. To the contrary, it is applicable to any tumor that consists of both M+ and MO forms and in which dydregulated genes can be identified. For example, the present invention is also applicable to other neurotumors, such as glioma, ependymoma and neuroblastoma, as well as to lung and breast cancers.

The embodiments shown below are designed merely to illustrate the invention. They are not to be construed as limiting the claims in any way.

### EXAMPLES

### Example 1

### Clinical Definition of Metastatic vs. Non-metastatic Disease.

Tumor samples were designated as metastatic (M+) if there was clinical documentation of tumor spread within the central nervous system (CNS) by MRI of the brain or spine at the time of diagnosis. Samples were not designated as M+ based solely on the presence of positive CSF cytology. The majority of patients did not have evaluations for metastatic disease outside the CNS. However, none of the M0 or M+ samples demonstrated clinical signs, symptoms, or laboratory evidence suggestive of metastatic disease outside the CNS at the time of diagnosis that would have routinely warranted further metastatic disease work-up such as bone scan or bone marrow evaluation by the treating clinician. Tumor samples were designated as non-metastatic (M0) if there was no evidence of tumor spread by any of the criteria above at the time of diagnosis and for at least 1 year from the time of diagnosis. We designated a single tumor as non-metastatic based on clinical parameters only, as radiological confirmation by MRI was not available. All tumor samples were obtained from the primary tumor site of the cerebellum.

### Example 2

### Treatment of Tumor Specimens

All tumor specimens used in this study were obtained with Institutional Review Board approval. Samples from each primary tumor were taken at the time of the initial diagnostic biopsy and were immediately frozen in liquid nitrogen and then continuously stored at −80° C. until used for array profiling. We immediately placed frozen specimens into TRIzol Reagent (Life Technologies, Inc.), homogenized them, and isolated total RNA as described. We also obtained an independent set of paraffin embedded tumor samples prepared at the time of the initial diagnostic biopsy from the archives of the pathology department of the Children's National Medical Center and were used for immunohistochemistry staining. Medulloblastoma cell lines Daoy, D341Med, and D283Med were grown in EMEM media (Biowhitaker) with 10% FBS at 37° C. with 5% CO₂ according to the specifications of the American Type Culture Collection (ATCC), and isolated total RNA as described.

### Example 3

### Expression Profiling

Total RNA from each medulloblastoma biopsy and cell line was isolated using TRIzol Reagent according to published procedures. Briefly, we obtained 10 to 25 ug of total RNA from each 0.5 cm² tumor. Initially, we converted 10 μg of RNA from each medulloblastoma into double-stranded CDNA with the SuperScript choice system (Life Technologies, Inc.) using an oligo-dT primer containing a T7 RNA polymerase promoter (Genset). We purified the double-stranded cDNA by phenol-choloroform extraction, which we used for in vitro transcription using the ENZO BioArray RNA transcript labeling kit (Affymetrix). The biotin-labeled cRNA was purified using the RNeasy kit (Qiagen),and subsequently fragmented the CRNA to approximately 200 bp by alkaline treatment (200 mM Tris-acetate, pH 8.2, 500 mM KOAc, 150 mM MgOAc). We verified the integrity of each labeled cRNA via hybridization to an Affymetrix TestChip2 array followed by detection with a streptavidin labeled fluor and confocal laser scanning according to the manufacturer's recommendations (Affymetrix). A labeled cRNA was deemed suitable if the ratio of the average intensity of the 3′ to 5′ ends of the beta-actin gene is less than 3. We subsequently hybridized each verified cRNA to a G110 Cancer Array (Affymetrix). The Affymetrix software extracted fluorescence intensities from each element on the arrays, representing 1992 human transcripts. We subsequently scaled the data from each array in order to normalize data for inter-array comparisons. We excluded from consideration all genes without “present” calls for any of the tumors, as determined by the Affymetrix software (leaving 1446 evaluable transcripts). To avoid division by zero or negative numbers in the calculation of average fold-differences between classes in FIG. 1, we set all average intensities of 10 or less to 10. For subsequent statistical methods, however, we used the complete unadjusted data set. We used the Cluster and TreeView software (downloaded from http://rana.lbl.gov

### Example 4

### Selection of Differentially Regulated Genes.

In order to reduce the dimensionality of the number of potential genes that could discriminate between classes in our dataset, we used a modification of current methods. Golub et al. (1999 above) describe a “distance metric” based on the ratio of the differences in class means divided by the sum of the class standard deviations. This metric works well for normally distributed data, but may be misleading when this assumption is not met.

Additionally, one must also specify in advance an arbitrary number of genes in order to choose a “cut point” for class prediction. We used permutational P values less than or equal to 0.05 based on permutation tests to select our candidate gene list. We performed permutation tests to determine which genes showed significant differences in mean intensity between the classes. In order to determine a permutational P value for each gene, we first calculated the mean and variances for the sample intensities within each class and calculated a t-test statistic (θ). We subsequently permuted the class assignments of the tumors, using sampling without replacement, . . . 50,000 times. For each permutation we calculated a test statistic θ*₍ᵢ₎ for all i=1 . . . , 50,000 permutations. The permutational P value is the probability of observing at least as large a value for the test statistic for any random permutation (θ*₍ᵢ₎) as for our initial test statistic (θ). This method has the benefit of not depending on distributional assumptions. It is important to note that we are aware of the potential problems for finding significant differences between classes when no difference actually exists. Current methods for adjustment for multiple comparisons, such as the Bonferroni adjustment (where the level of significance, a, is divided by the number of tests, in this case 2059) break down quickly in the microarray setting. We contend that since we are using this technique as an exploratory measure to reduce dimensionality, the application of multiple comparison correction is not a requirement.

### Example 5

### Prediction Methodology

We adapted the methodology of Golub et al. (1999, above) to classify samples based on the set of “predictor” genes with a permutational P<0.05. Briefly, each gene gi votes for either the metastatic or non-metastatic class, depending on whether the expression level xᵢ in the sample is closer to the mean expression level of the metastatic (μ_{M+}) or non-metastatic (μ_{M0}) class of reference samples. The magnitude of the vote (vᵢ) reflects the deviation of the expression level in the sample from the average of the two classes:

vᵢ=|xᵢ−(μ_{M0}+μ_{M+})/2|

The votes are summed in order to obtain total votes for the non-metastatic (V_{M0}) and metastatic (V_{M+}) classes. Prediction strength values are then calculated as follows:

Prediction Strength=|(V_{M0}−V_{M+})/(V_{M0}+V_{M+})|

Prediction strength values reflect the margin of victory in the direction of either the non-metastatic or metastatic class. Prediction strength ranges from 0 to 1, with higher values reflecting stronger predictions. We assigned samples to the winning class only if the prediction strength for that sample exceeded the prediction strength threshold of 0.23. Our value of 0.23 was scaled to be equivalent with the threshold of 0.3 used by Golub et al. (1999, above) for their 50 gene predictor, as prediction strength scales inversely with the square root of the number of genes in the predictor.

Predictions were generated for the original 23 non-metastatic and metastatic samples using a leave-one-out methodology. Samples were removed one at a time from the sample set, permutational statistics and expression means were recalculated for each class for the modified sample set, and predictions generated as above using only genes with a permutational P<0.05 in the modified sample set. Scaling the prediction threshold for the exact number of genes in each individual cross-validation predictor had no effect on the prediction accuracy obtained.

We calculated votes for the five validation tumors and the cell lines using mean expression levels and a gene list (genes with permutational P<0.05) derived from the complete set of 23 tumors.

### Example 6

### Immunohistochemistry

Sections (4 μm) of formalin-fixed paraffin tissue were cut with a microtome and mount to gelatin-coated slides. Slides were incubated on a warming table at 42 ° C. for 15 min. We subsequently deparaffinized the tissue sections in three xylene changes for 5 min each, and hydrated them via two washes in 100% ethanol (10 min each), two washes in 95% ethanol (10 min each), and one wash in deionized water (1 min). We used DAKO Target Retrieval Solution for antigen unmasking according to the protocol of the manufacturer (DAKO Corporation). We incubated the sections with 1% hydrogen peroxide to quench endogenous peroxidase activity, and washed them twice in in PBS (2 min each wash).

Sections were incubated in 100 μl of diluted primary antibody for 2 h in a humidified chamber. We diluted primary antibodies with 1.5% BSA in PBS as follows: PDGFRA polyclonal rabbit anti-human (Santa Cruz Biotechnology), 1:50; SPARC monoclonal mouse anti-human (Zymed Laboratories), 1:200; PCNA monoclonal mouse anti-human (Santa Cruz Biotechnology), 1:200. Following incubation with primary antibody, we washed the sections three times in PBS (1 min each). We subsequently incubated sections with diluted secondary antibody for 1 h. We diluted biotinylated secondary antibodies with 1.5% BSA in PBS as follows: goat anti-mouse (Santa Cruz Biotechnology), 1:500; and goat anti-rabbit (Santa Cruz Biotechnology), 1:300. We incubated sections with 1-2 drops of HRP substrate (Santa Cruz Biotechnology) for 20 min, and subsequently washed them in deionized water for 2 min. We counter-stained the sections in Gill?s formulation #2 hematoxylin for 5 sec and immediately washed them 7-10 times in deionized water. Finally, we dehydrated the sections with two washes in 95% ethanol, two washes in 100% ethanol, and three washes in xylene (all washes were 10 see). We added glass coverslips to the sections using Permount. We photographed all sections on a conventional light microscope at 200X.

Two individuals independently reviewed the slides and subjectively scored them for the absence or presence of weak (+), intermediate (++), or strong (+++) staining. We compared the number of positively ( +, ++, and +++ combined) and negatively (−) stained slides for metastatic and non-metastatic tumors for all three proteins using a Fisher's exact test.

### Example 7

### In Vitro Motility and Adhesion Assays.

Fibronectin-mediated cell adhesion and migration were assessedusing the Chemicon QCM-FN Quantitative Cell Migration Assay (Chemicon International). Briefly, Daoy cells were grown in EMEM withh 10% FBS to 80% confluency and starved in serum-free EMEM for 24 h. Cells were washed twice with PBS, harvested them with trypsin/EDTA, and resuspended them in serum-free EMEM (1×10⁶ cells/ml) with 5% BSA for 1 h. We subsequently incubated cells at 37 ° C. for 30 min with either no antibody, 0.6 ug/ml of neutralizing monoclonal anti-human PDGFRA antibody (R&D Systems), 0.6 ug/mi isotype-matched control antibody (R&D Systems), and or first pretreated them with 10 μM of MAP2K1/MAP2K2 inhibitor U0126 (Cell Signaling Technology) for 1 h. We based the concentration of antibody used on the ND₅₀ for this lot of antibody for blocking 10 ng/ml of PDGFRA biologic activity on human WS1 cells (per R&D Systems product insert). We based the concentration of MAP2K1/MAP2K2 inhibitor U0126 used on the manufacturer?s recommendation. Following incubations, we resuspended the cells, and placed aliquots of 2×10⁵ cells in 200 ul of serum-free EMEM into the upper fibronectin-(FN-) coated or BSA-coated (control) Boyden chambers (migration assay), or FN-coated wells (adhesion assay). The lower well contained 0, 1, 2, 4, 6, 8, or 10 ng/ml PDGFA (Chemicon) in 300 \grave{l}l serum-free EMEM. We incubated the cells for 3 h at 37° C., stained them with cell stain solution (Chemicon), and subsequently washed them. We removed the medium, as well as non-adherent or non-migrating cells, from the upper side of the Boyden chamber membranes. We first visualized the cells by microscopy, and then subsequently eluted migrating or adherent cells with extraction solution (Chemicon) according to the manufacturer? s instructions. We aliquoted eluant (50 μl)×2 for each test sample into microtiter plates, and read the optical density (OD₅₄₀₋₅₇₀) on a ThermoMax microplate reader (Molecular Devices). Using standardized migration rates of 130 cells per mm² equivalent to an OD₅₅₀ of 0.270 (per Chemicon product insert), we obtained the final cell counts by averaging the two OD measurements (540 nm and 570 nm) after subtraction of the BSA control measurements (background), and determined significance (P value) using a t-test.

### Example 8

### Western Blot

Daoy cells were treated in a manner identical to that described above for migration. After described incubation with PDGFA, PDGFRA antibody, or MAP2K1/MAP2K2 inhibitor U0126, we prepared cell lysates by adding 500 ul lysis buffer (Cell Signaling Technology) to cell pellets on ice for 20 min, sonicating, and centrifuging at 10,000 RPM for 10 min. We collected the supernatants and mixed equal amounts of lysate protein (5.2 ug) in a 1:1 mixture with Tris-Glycine SDS sample buffer Covex). Samples were run on a 12% SDS-PAGE gel (BioRad) along with broad range protein markers (BioRad) at 125 V for 1 h. We transferred proteins to PVDF membrane (NEN Life Sciences) using a semi-dry transfer cell (BioRad). We first soaked the membrane in anhydrous methanol for 2 min, deionized water for 5 min, and transfer buffer (25 mM Tris, 0.2 M glycine, 20% methanol, pH 8.5) for 10 min. The gels were pre-soaked in transfer buffer for 15 min and subsequently electro-blotted to the membrane between filter paper soaked in transfer buffer for 45 min at a current equivalent to 2.5 × total gel area (cm²). Following blotting, we incubated the membrane in blocking buffer (3% BSA in TBS-T) for 1 h at room temperature with gentle agitation. We subsequently incubated the membrane with primary antibody (MAP2K1/2 antibody, phospho-MAP2K1/2 (Ser217/221) antibody, MAPK1/3 (p42/p44 MAPK) antibody, or phospho-MAPK1/3 (Thr202/Tyr204) antibody; Cell Signaling Technology) diluted 1:1000 in blocking buffer overnight at 4° C. We washed the membranes in wash buffer (1% BSA in TBS-T) 3 times (1× for 10 min and 2× for 5 min) and incubated them with anti-rabbit HRP-conjugated secondary antibody (Cell Signaling Technology) diluted 1:2000 in blocking buffer for 1 h with gentle agitation at room temperature. We subsequently washed membranes with gentle agitation 5 times with 1%BSA in TBS-T (1× for 10 min in, 4× for 5 min) and once in TBS-T for 5 min. We used LumiGlo for detection (0.5 ml 20× LumiGlo, 0.5 ml 20× peroxide and 9.0 ml water; Cell Signaling Technology) with gentle agitation for 2 min at room temperature. We removed membranes from the developer, wrapped them in plastic wrap, and exposed them to BioMax film (Kodak) for 2 sec. We scanned the developed films, and determined the optical density of each lane using NIH Image (available at http://rsb.info.nih.gov/nih-image/Default.html). We used the ratio of phspho-MAP2K1/2 and MAPK1/3 to total MAP2K1/2 and MAPK1/3 for each lane to determine the relative change in phosphorylation for each protein.

### Example 9

### Identification of Predictor Genes for Class Assignment

Twenty-three medulloblastoma tumors (10 metastatic and 13 non-metastatic) were expression profiled on individual Affymetrix G110 arrays. We chose the G110 arrays as they are enriched for genes that have been implicated in cancer biology. After extraction of the fluorescent intensities for each gene using the GeneChip software, we normalized each data set to facilitate inter-array comparisons.

We used a two-step method to identify genes differentially expressed between metastatic and non-metastatic tumors. We first used permutation statistics to identify “candidate genes” which differ in expression between metastatic and non-metastatic tumors. We subsequently adapted a weighted voting class-prediction algorithm to validate our set of genes by blindly predicting the class of our tumor samples using a leave-one out approach. For each probe set on the array, we obtained a permutational P value by calculating a t-statistic for the two classes of tumors, as well as for 50,000 random permutations of the data. We selected all genes with a permutational P value less than 0.05 as candidate “predictor” genes. 59 genes showed increased expression in metastatic tumors, while 26 showed decreased expression in metastatic tumors (FIG. 1).

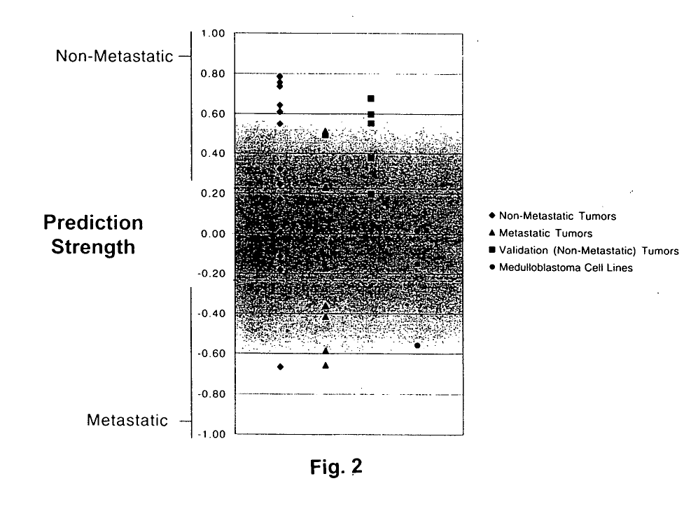

Using this gene list and the data from the 23 medulloblastomas, we adapted the methodology of Golub et al. (1999, above) to diagnose samples that were blinded as to phenotype. For any given sample, each gene casts a “weighted vote” which is dependent on the expression level as compared to the mean expression levels of the two sample classes. We summed the votes to determine both the winning class and the prediction strength, a measure of the margin of victory ranging from 0 to 1. We assigned samples to the winning class only if the prediction strength exceeded a threshold (0.23). We classified samples with prediction strengths below this threshold as uncertain. All 23 of our tumors were predicted using a leave-one-out approach, in which one sample is withheld, permutational P values and mean expression levels were calculated using the remaining samples, and the class of the withheld sample is subsequently predicted as above using only the genes with a recalculated permutational P<0.05 (FIG. 2). Of the 23 tumors, the predictor assigned 18 to either the metastatic or non-metastatic class, while not classifying the remaining five. The predictor classified 90% of non-metastatic tumors correctly (9/10), while classiflying 50% of metastatic tumors correctly (4/8).

As a means to independently test our prediction method using the gene list generated from the complete dataset of all 23 tumors, we profiled five additional tumors blinded by M-status at diagnosis and applied the prediction algorithm (FIG. 2). The predictor assigned four of the five tumors to a sample class. All four of these predictions were consistent with the sample diagnosis. Finally, we profiled three metastatic medulloblastoma cell lines and applied the predictor (FIG. 2). We classified the two adhesion-independent cell lines (D283Med and D341Med cells) as “uncertain” while classifying the adhesion-dependent Daoy cell line as metastatic with prediction strength of 0.56.

### Example 10

### Immunohistochemical Validation of Critical Pathway Members

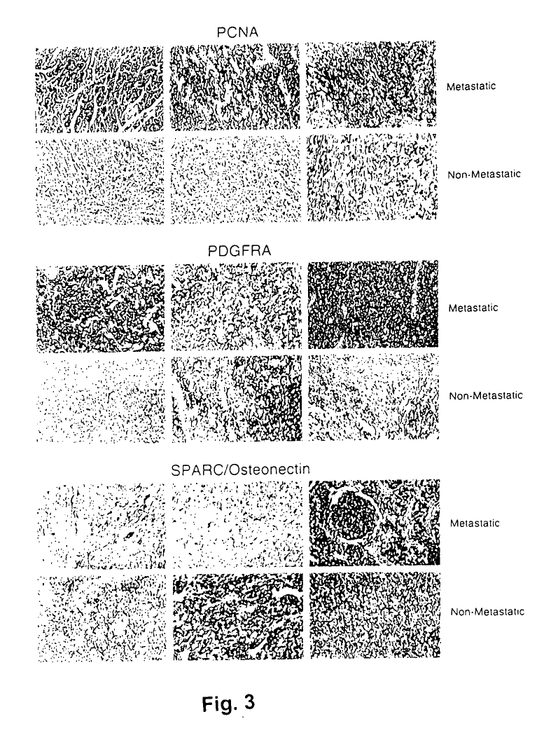

We performed immunohistochemistry for PDGFRA and SPARC (secreted protein a acidic and rich in cysteine), an anti-adhesive and pro-angiogenic matricellular glycoprotein, in an independent set of paraffin embedded tumors consisting of six metastatic and 14 non-metastatic tumors (FIG. 3). We also performed immunohistochemistry for proliferating cell nuclear antigen (PCNA), an auxiliary protein to DNA polymerases delta and epsilon implicated in DNA replication and repair, in three metastatic and three non-metastatic tumors. A summary of the staining results in Table 1 shows that for PDGFRA, 83% of M+ tumors stained positive, whereas 0% of M0 tumors stained positive (P=0.00004). For SPARC, 100% of the M+ tumors were positive, while only 15% of M0 tumors were positive (P=0.0025). Furthermore, none of the M0 tumors tested stained positive for more than one of the three proteins evaluated, while all of the M+ tumors, in which staining was performed for at least two proteins, stained positive for each of the two or three proteins evaluated.

We observed no histopathological differences between the M+ and M0 tumors by H&E staining. Of note was the heterogeneity of the immunoperoxidase staining in the M+ set of tumors. For example, some sections within the M+ tumors clearly demonstrated higher levels of PDGFRA expression (“hot spots”) than other areas; however, there appeared to be no correlation with other histologic features, such as vascularity. Only infrequently were there areas of the M+ tumors that appeared negative for staining. In contrast, the M0 tumors were uniformly low-to-negative staining throughout, with only infrequent “hot spot”, areas that were generally less intense than those observed in M+ tumors.

TABLE 1
Immunohistochemistry results for 6 metastatic
and 14 non-metastatic tumors stain a, b

| M-status | PDGFRA | SPARC | PCNA |
|---|---|---|---|
| metastatic | +++ | +++ | +++ |
| metastatic | +++ | +++ | +++ |
| metastatic | ++ | ++ | ++ |
| metastatic | ++ | + | nd |
| metastatic | + | + | nd |
| metastatic | − | ne | nd |
| non-metastatic | − | − | − |
| non-metastatic | − | − | − |
| non-metastatic | − | − | + |
| non-metastatic | − | + | nd |
| non-metastatic | − | ne | nd |
| non-metastatic | − | − | nd |
| non-metastatic | − | − | nd |
| non-metastatic | − | − | nd |
| non-metastatic | − | + | nd |
| non-metastatic | − | − | nd |
| non-metastatic | − | − | nd |
| non-metastatic | − | − | nd |
| non-metastatic | − | − | nd |
| non-metastatic | − | − | nd |
| % positive, | 83 (5/6) | 100 (5/5) | 100 (3/3) |
| metastatic | | | |
| % positive, | 0 (0/14) | 15 (2/13) | 33 (1/3) |
| non-metastatic | | | |
| P-value(c) | * 0.00004 | * 0.0025 | 0.4 |

Formalin-fixed, paraffin-embedded tumors were stained with antibodies against PDGFRA, SPARC, and PCNA(a),
“−,, denotes undetectable stain;
“+”: weak staining,
“++”: intermediate staining;
“+++”: strong staining;
“ne”: tumor section not evaluable; and
“nd”: staining not done
“b”. Significance of differences in positive/negative staining (*) between tumor classes was determined using a Fisher's exact test(c).

### Example 11

### PDGFRA-Blocking Antibodies and Ablation of the Metastatic Phenotype In Vitro

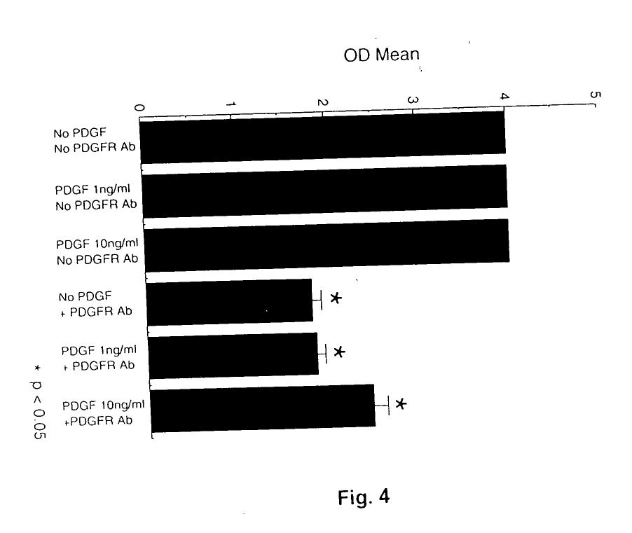

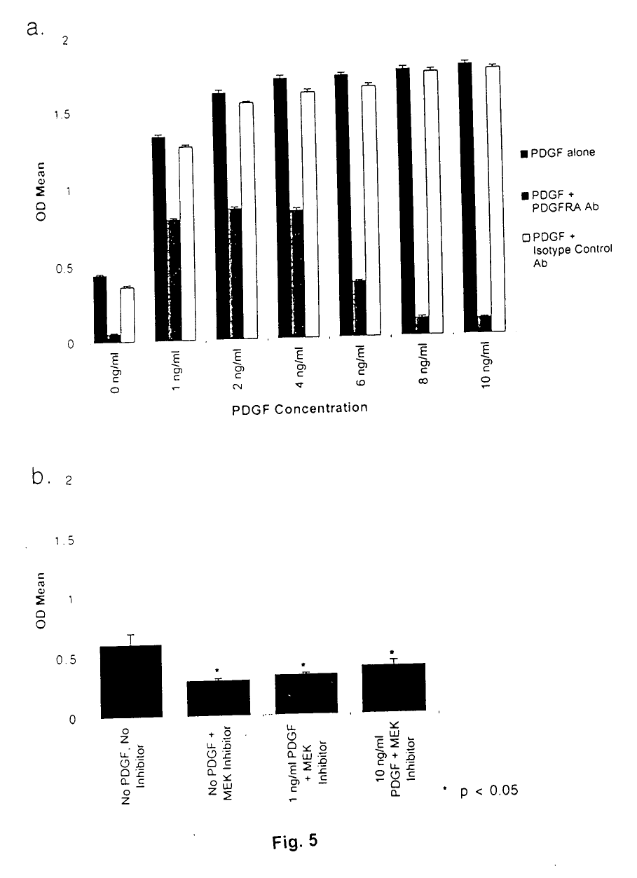

We tested the significance of PDGFRA in the metastatic potential of Daoy cells by fibronectin-dependent adhesion and migration assays. Incubation with increasing concentrations of PDGFA did not affect the adhesion of Daoy cells to fibronectin; however, blocking the PDGFA receptor with monoclonal antibodies inhibited adhesion to 45% normal (FIG. 4). Incubation of the cells with 1 ng/ml PDGFA added to the medium did not alter the adhesion inhibition induced by pre-treating the cells with anti-PDGFRA antibody; however, 10 ng/ml reduced the antibody-mediated inhibition to 61% normal in a manner most characteristic of competitive inhibition. In contrast to the responses observed in the adhesion assays, the addition of 1 ng/ml PDGFA to the lower wells of the Boyden chambers significantly stimulated Daoy migration across fibronectin-coated membranes by approximately 3-fold (640 vs. 212 cells/mm²). The increased migration stimulated by PDGFA was dose-dependent, reaching near-maximum levels around 4 ng/ml (819 vs. 212 cells/mm²) and leveling out at a 4-fold increase in migration at 10 ng/ml (847 vs. 212 cells/mm²) (FIG. 5a). Even more striking was the ablation of baseline migration following treatment of cells with neutralizing antibody to PDGFRA. Treatment of cells with PDGFRA neutralizing antibody blocked cell migration nearly completely in the absence of PDGFA compared to untreated cells (24 vs 212 cells/mm²). Addition of 1 ng/ml PDGFA to the lower wells of the Boyden chambers containing antibody-treated cells resulted in recovery of migration to 60% of the expected level (380 vs. 640 cells/mm²). Surprisingly, increasing concentrations of PDGFA above 1 ng/ml in the lower wells of antibody-treated cells did not further stimulate the recovery of migration in these cells to the expected levels in non-treated cells. Instead antibody-treated cells incubated with concentrations of 2 ng/ml PDGFA showed diminished cell migration (52% of expected), with a near complete arrest of migration (6% of expected) in antibody-treated cells incubated with 10 ng/ml PDGFA (847 vs. 48 cells/mm²). Treatment with the isotype-matched control antibody did not significantly diminish expected migration at any concentration of PDGFA tested, suggesting that the PDGFRA neutralizing antibody is indeed specific for PDGFRA.

### Example 12

### PDGFRA Blocking Antibodies Inhibit MAP2K1/2 and MAPK1/3 Activation

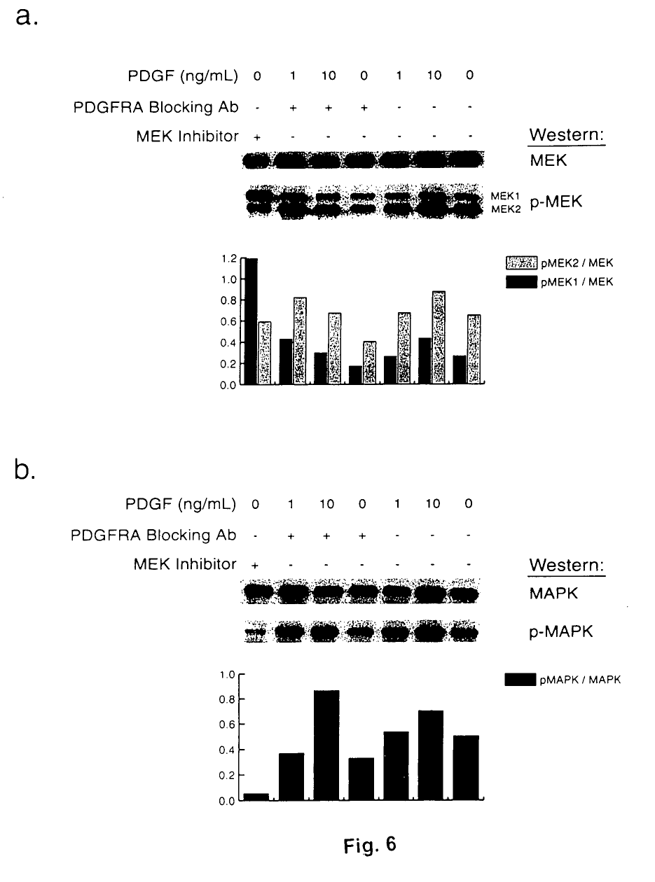

To determine whether the inhibition of cell migration induced by the PDGFRA blocking antibodies correlates with the loss of downstream signaling activation, we performed Western blot analysis using phospho-specific antibodies to MAP2K1/2 and MAPK1/3 on cell lysates prepared from Daoy cells that had been treated in an identical fashion as that described above and tested at the time point of the start of the migration assay (FIG. 6a,b). PDGFA (10 ng/ml) caused 1.6-fold, 1.3-fold, and 1.7-fold increases in MAP2K1, MAP2K2, and MAPK1/3 phosphorylation, respectively. PDGFRA blocking antibodies resulted in 1.5-fold, 1.6-fold, and 1.5-fold reductions in baseline phosphorylation of MAP2K1, MAP2K2, and MAPK1/3, respectively. Interestingly, pre-treatment with PDGFRA blocking antibodies prevented PDGFA enhancement of MAP2K1 and MAP2K2 phosphorylation, but not MAPK1/3 phosphorylation.

### Example 13

### MAP2K1/2 Inhibitor U0126 Blocks Migration in Vitro

To further test whether medulloblastoma migration is directly linked in part to downstream MAP2K1, MAP2K2 and MAPK1/3 activation, Daoy migration was tested in an identical fashion to that described above, except that cells were pretreated with U0126, a highly specific chemical inhibitor of MAP2K1 and MAP2K2 (FIG. 5b). U0126 caused a 9-fold decrease in baseline phosphorylation of MAPK1/3 at the time of the start of the migration assay (FIG. 6b). U0126 treated cells showed 50% decreased cell migration (275 vs. 139 cells/mm², P<0.05). Addition of 1 ng/ml and 10 ng/ml PDGFA to the U0126 treated cells resulted in recovery of migration to 57% (275 vs. 158 cells/ mm², P<0.05) and 65% (275 vs. 188 cells/ mm², P<0.05) of normal baseline migration, respectively.

We claim:

1. A gene-based method for predicting metastasis in a tumor that exists in both metastatic (M+) and non-metastatic (MO) classes, comprising the steps of:
A. Identifying by expression-profiling of tumor sample cohorts of said M+ and MO classes of said tumor, coupled with permutational statistical analysis to generate a candidate gene list, those genes whose expression differ statistically between said classes of said tumor and that are upregulated in the M+ class and downregulated in the MO class;
B. producing a class-predictive algorithm based upon said predictive genes with a permutational P value of<0.05; and
C. applying said algorithm to a candidate tumor to produce a Predictive Strength value that will assign the M+ or MO class to said tumor.

2. The method according to claim 1, wherein said expression profiling is carried out using microarrays of oligonucleotide gene chips.

3. The method according to claim 1, wherein said tumor is a neurotumor.

4. The method according to claim 3, wherein said tumor is a medulloblastoma.

5. The method according to claim 3, wherein said tumor is a glioma.

6. The method according to claim 3, wherein said tumor is a neuroblastoma.

7. The method according to claim 3, wherein said tumor is an ependymoma.

8. The method according to claim 1, wherein said tumor is lung cancer.

9. The method according to claim 1, wherein said tumor is breast cancer.

10. The method according to claim 4, wherein said predictive M+ genes that are up-regulated in said metastatic tumor are found in the group consisting of: invasion and angiogenesis genes, growth factor or cytokine-mediated proliferative genes, signal transduction genes, transcriptional regulatory genes, DNA duplicative genes, and oncogenesis genes.

11. The method according to claim 4, wherein said predictive upregulated M+ genes and said predictive downregulated MO genes are as listed in listed in FIG. 1.

12. The method according to claim 4, wherein said predictive gene comprises at least one of the M+ gene group consisting of PDGFRA, FGFR2, IGFBP2, IGFBP7, RAS/MAPK pathway, PDGFA, ITGA4, ITGB5, SPARC, TIMP1, TIE, HOXA4, HOXA7, NTRK3, MYC, CTSC, CTSD, BLM, TPBG and MSH2, as these genes are defined in the specification.

13. The method according to claim 12, wherein said upregulated predictive M+ gene is the gene for PDGFRA.

14. The method according to claim 12, wherein said upregulated predictive M+ gene is a member of the downstream RAS/mitogen-activated protein kinase (MAPK) signal transduction pathway.

15. The method of claim 13, wherein said PDGFRA M+ gene enhances medulloblastoma migration and upregulates at least one member of the MAPK group of genes.

16. The method according to claim 1, wherein said algorithm comprises two primary equations:
vᵢ=[xᵢ−(μM₀+μ_{M+})/2] (1)
wherein vi is the selective vote, xi is the expression level in the tumor sample, and μMO and μM+ are the metastatic classes of reference samples, and wherein said votes are summed in order to obtain total votes for the non-metastatic (V_{M0}) and metastatic (V_{M+}) classes; and,
Prediction Strength=[(V_{M0}−V_{M+})/(V_{M0}+V_{M+})] (2)
wherein Prediction Strength values range between 0 and 1.

17. The method according to claim 10, wherein said Prediction Strength is no less than 0.23.

18. A method for inhibiting or reversing in vivo metastisis in a M+ class tumor in a subject, comprising the step of administering to said subject an effective amount and for an effective period of time an inhibitor of the upregulation (overexpression) of a gene identified by the method of claim 1 as being associated with said M+ class.

19. The method according to claim 18, wherein said inhibitor is a neutralizing antibody directed against the protein encoded by said upregulated M+ gene.

20. The method according to claim 18, wherein said inhibitor is a chemical inhibitor.

21. The method according to claim 20, wherein said inhibitor is directed against a member of the the metastatic overexpressed gene group consisting of the signal transduction inhibitor STI-571, the RAS inhibitor R115777, the MAP2K1/MAP2K2 protein kinase inhibitor U0126, the specific signal transduction inhibitor of PDGFRA STI-571, the phosphoinositide 3-kinase inhibitor wortannin, the VEGF inhibitor NM3, the MAP kinase inhibitor CC1-779, and the glutathione S-trandferase inhibitor TLK 886.

22. The method according to claim 21, wherein said inhibitor is the RAS inhibitor R115777.

23. The method according to claim 21, wherein said inhibitor is SCH88336.

24. The method according to claim 21, wherein said inhibitor is U0126.

25. The method according to claim 21, wherein said inhibitor is STI-571.
